(19) United States
(12) Patent Application Publication
(10) Pub. No.: US 2011/0301428 A1
(43) Pub. Date: Dec. 8, 2011

(54) Lightweight automatic gain control for ambulatory monitoring systems

(76) Inventors: Yongji Fu, Vancouver, WA (US); Bryan Severt Hallberg, Vancouver, WA (US); Bharat Kumar Vegesna, Vancouver, WA (US)

(21) Appl. No.: 12/802,336

(22) Filed: Jun. 4, 2010

(51) Int. Cl. A61B 5/00; H03L 5/00

(52) U.S. Cl. 600/300; 327/306

(57) ABSTRACT

Lightweight automatic gain control (AGC) methods and systems reduce usage of often scarce computing resources in ambulatory monitoring systems through an AGC algorithm that relies on lightweight calculations and judicious constraints on gain reevaluations and adjustments. Statistical range sampling is used to adjust the gain of a physiological signal to keep the signal within a target amplitude range and may be coupled with dynamic range control to prevent gain adjustments from occurring too frequently. Moreover, gain reevaluations and adjustments may be temporarily suspended when the physiological signal is noisy.

### BACKGROUND OF THE INVENTION

The present invention relates to ambulatory monitoring and, more particularly, to lightweight automatic gain control (AGC) methods and systems designed to improve ambulatory monitoring systems.

Ambulatory monitoring of the physiological state of people who suffer from chronic diseases is an important aspect of chronic disease management. By way of example, ambulatory monitoring is in widespread use managing chronic diseases such as asthma and in elder care.

Ambulatory monitoring is often performed using a portable (e.g. wearable) device that continually acquires and analyzes physiological signals, such as a signal that includes heart and lung sounds, as a person wearing the device goes about his or her daily life. The level of the physiological signal acquired by the device can vary widely depending on individual attributes of the person being monitored and also due to changes in background noise, the person's current activity level (e.g. idle, walking, running) and the current device function. AGC technology may be deployed on the device to continually reevaluate and adjust the gain of the physiological signal to ensure the signal amplitude remains within a target range, and does not become saturated or too small.

AGC technologies deployed in ambulatory monitoring systems may perform sub-optimally for several reasons. Sometimes, the AGC technology consumes an unacceptably large share of computing resources, which are limited on a portable device. Resource utilization issues may be magnified when frequent gain reevaluation is required due to, for example, rapid changes in background noise or user activity. Additionally, the AGC technology may be unable to detect when a physiological signal is too unreliable to warrant gain reevaluation and adjustment. For example, a physiological signal may be too noisy to reliably recover physiological data when a person speaks, or may be too weak to reliably recover physiological data when a person does not place a physiological sensor at a proper body location. In these situations, confidence in physiological data that could be extracted from the physiological signal, such as the patient's heart rate, may be sufficiently low that it would be preferable to output an indication that the signal is unreliable, rather than consuming scarce computing resources to reevaluate and adjust the gain of the unreliable signal.

### SUMMARY OF THE INVENTION

The present invention provides lightweight AGC methods and systems for ambulatory monitoring systems. The invention conserves often scarce computing resources in ambulatory monitoring systems through an AGC algorithm that relies on lightweight calculations and judicious constraints on gain reevaluations and adjustments. Statistical range sampling is used to adjust the gain of a physiological signal to keep the signal within a target amplitude range and may be coupled with dynamic range control to prevent gain adjustments from occurring too frequently. Moreover, gain reevaluations and adjustments may be temporarily suspended when the physiological signal is noisy.

In one aspect of the invention, an AGC method for an ambulatory monitoring system comprises the steps of continually monitoring, by the system, for gain reevaluation events; reevaluating, by the system, a gain of a physiological signal in response to a gain reevaluation event, including sampling the physiological signal to obtain a first plurality of samples, determining a share of the first plurality of samples that is outside a target amplitude range and comparing the share with a first limit share; and adjusting, by the system, the gain based at least in part on a determination that the share exceeds a first limit share.

In some embodiments, the adjusting step includes setting the gain to a minimum, sampling the physiological signal to obtain a second plurality of samples, determining shares of the second plurality of samples that are within respective gain-specific amplitude domains and adjusting the gain to a level associated with a gain-specific amplitude domain for which a share of the second plurality of samples exceeds a second limit share.

In some embodiments, the gain reevaluation events include function change events. In some embodiments, the gain reevaluation events include motion sensor events. In some embodiments, the gain reevaluation events include periodic signal check events.

In some embodiments, the AGC method further comprises the step of adjusting, by the system, the target amplitude range in response to a determination that a gain adjustment rate exceeds a limit rate.

In some embodiments, the AGC method further comprises the step of temporarily suspending, by the system, the gain reevaluating and adjusting steps in response to a determination that a noise level of the physiological signal exceeds a limit noise level.

In another aspect of the invention, an AGC method for an ambulatory monitoring system comprises the steps of reevaluating, by the system, a gain of a physiological signal including comparing the physiological signal with a target amplitude range; adjusting, by the system, the gain in response to the comparison; and adjusting, by the system, the target amplitude range in response to a determination that a gain adjustment rate exceeds a limit rate.

In another aspect of the invention, an AGC method for an ambulatory monitoring system comprises the steps of reevaluating, by the system, a gain of a physiological signal including comparing the physiological signal with a target amplitude range; adjusting, by the system, the gain in response to the comparison; and temporarily suspending, by the system, the gain reevaluating and adjusting steps in response to a determination that a noise level of the physiological signal exceeds a limit noise level.

In some aspects, these method steps of the invention are performed by a physiological data acquisition system of an ambulatory monitoring system, wherein the ambulatory monitoring system comprises a physiological data capture system, the acquisition system communicatively coupled with the capture system, a physiological data processing system communicatively coupled with the acquisition system and a physiological data output interface communicatively coupled with the processing system.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows an ambulatory monitoring system in some embodiments of the invention.

FIG. 2 shows gain reevaluation event monitoring performed in an ambulatory monitoring system in some embodiments of the invention.

FIG. 3 shows a profile of a physiological signal in some embodiments of the invention.

FIG. 4 shows statistical range sampling performed in an ambulatory monitoring system attendant to gain reevaluation to obtain data for use in a signal range check, in some embodiments of the invention.

FIG. 5 shows a signal range check performed in an ambulatory monitoring system attendant to gain reevaluation to determine whether gain adjustment is warranted, in some embodiments of the invention.

FIG. 6 shows gain adjustment performed in an ambulatory monitoring system in some embodiments of the invention.

FIG. 7 shows dynamic range control performed in an ambulatory monitoring system in some embodiments of the invention.

FIG. 8 shows temporary gain reevaluation suspension performed in an ambulatory monitoring system in some embodiments of the invention.

### DETAILED DESCRIPTION OF A PREFERRED EMBODIMENT

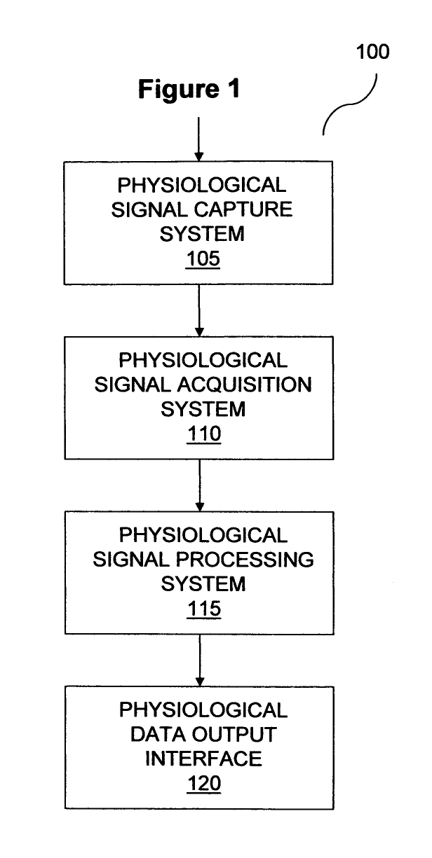

FIG. 1 shows an ambulatory monitoring system 100 in some embodiments of the invention. Monitoring system 100 includes a physiological signal capture system 105, a physiological data acquisition system 110, a physiological data processing system 110 and a physiological data output interface 115 communicatively coupled in series.

Capture system 105 detects physiological sounds at a detection point, such as a trachea, chest or back of a person being monitored and transmits a physiological signal to acquisition system 110 in the form of an electrical signal generated from detected physiological sounds. Capture system 105 may include, for example, a sound transducer positioned on the body of a human subject. In other embodiments, another type of transducer, such as an electrical (e.g. electrocardiogram) or optical transducer, may be used to capture a physiological signal.

Acquisition system 110 amplifies, filters, performs analog/digital (ND) conversion and AGC on the physiological signal received from capture system 105. Amplification, filtering and ND conversion may be performed by serially arranged pre-amplifier, band-pass filter, final amplifier and ND conversion stages. Acquisition system 110 performs AGC on the digitized physiological signal under control of a processor adapted to execute software to produce a gain-adjusted physiological signal without impacting on signal-to-noise ratio, and transmits the gain-adjusted signal to processing system 115. Gain adjustments are made to ensure that the amplitude of the physiological signal remains within a target amplitude range and does not become saturated or too small.

Processing system 115 under control of the processor performs time domain signal processing on the physiological signal to classify the signal and/or extract data from the signal. Processing system 115 under control of the processor outputs information generated based on the physiological signal to data output interface 120.

Data output interface 120 includes one or more of a user interface, a local analysis module, a data management element and a network interface for displaying, processing, storing and/or transmitting information generated based on the physiological signal and received from processing system 115.

Capture system 105, acquisition system 110, processing system 115 and data output interface 120 may be collocated or located remotely from one another and may be communicatively coupled via wired or wireless links. In some embodiments, capture system 105, acquisition system 110, processing system 115 and data output interface 120 are part of a portable (e.g. wearable) device that monitors a person's physiological state in real-time as the person performs daily activities.

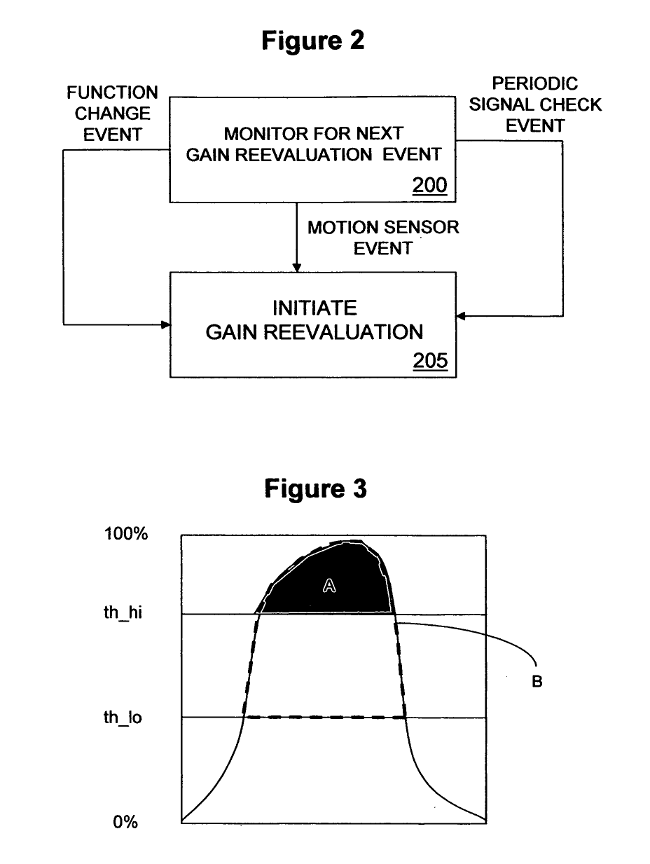

FIG. 2 shows gain reevaluation event monitoring performed by acquisition system 110 under processor control in some embodiments of the invention. During operation of monitoring system 100, acquisition system 110 continually monitors for gain reevaluation events (200), which include function change events, motion sensor events and signal range violation events. Initialization of monitoring system 100 also triggers gain evaluation. A function change event is detected in response to a change to a designated system function. For example, if a system function becomes operative that requires the person being monitored to do forced breathing, the signal level can be expected to become elevated and the gain may need to be adjusted downward keep the signal within the target amplitude range and avoid saturation. A motion sensor event is detected in response to a significant change in the measured motion of monitoring system 100. For example, monitoring system 100 may have an accelerometer that measures the current activity level of the person being monitored (e.g. idle, walking, running). A significant change in the activity level can be expected to cause a substantial change in the signal level and gain may need to be adjusted upward or downward to keep the physiological signal in the target amplitude range. A periodic signal check event is detected in response to expiration of a periodic signal check timer. In this regard, a periodic signal check is normally performed for a sampling period t, within every larger period t₀. Upon detection of any of the foregoing events, acquisition system 110 initiates reevaluation of the gain of the physiological signal (205) and continues monitoring for the next gain reevaluation event (200).

FIG. 3 shows a profile of a physiological signal on acquisition system 110 after application of a gain in some embodiments of the invention. In the profile, a target amplitude range is bounded by a high amplitude threshold th_hi and a low amplitude threshold th_lo. When the amplitude of the physiological signal is above the high threshold, the signal is in a domain A. When the amplitude of the physiological signal is above the low threshold, the signal is in a domain B. These thresholds and domains are used in lightweight statistical range sampling to keep the physiological signal within the target amplitude range. Particularly, if over a given sampling period the share of signal amplitude samples in domain A exceeds a limit share P_{A}, the physiological signal is deemed to have significant saturation risk and warrants downward gain adjustment. If over a signal range check the share of signal amplitude samples in domain B is below a limit share P_{B}, the physiological signal is deemed to be too weak and warrants upward gain adjustment. By way of example, P_{A} may be set at 5% and P_{B} may be set at 95%. In general, P_{A} P_{B}=1.

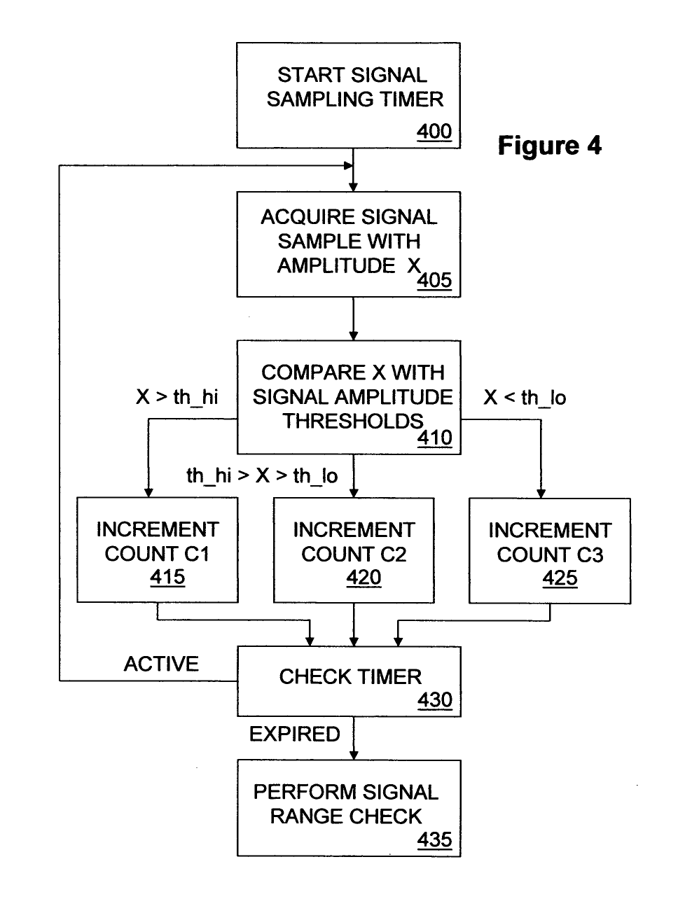

FIG. 4 shows statistical range sampling performed by acquisition system 110 under processor control attendant to gain reevaluation to obtain data for use in a signal range check, in some embodiments of the invention. At the beginning of the sampling period, acquisition system 110 starts a signal sampling timer (400) and acquires a first sample of the physiological signal, which sample has an amplitude X (405). The sample amplitude X is compared with the high amplitude threshold th_hi and low amplitude threshold th_lo (410). If the sample amplitude X is above th_hi, a first count C1 is incremented (415). If the sample amplitude X is between th_hi and th_low, a second count C2 is incremented (420). If the sample amplitude X is below th_lo, a third count C3 is incremented (425). In any case, the timer is checked (430) and if the sampling period is still active, the flow returns to Step 405 and processes another sample. If the sampling period has expired, a signal range check is performed using the counts obtained in the sampling period (435).

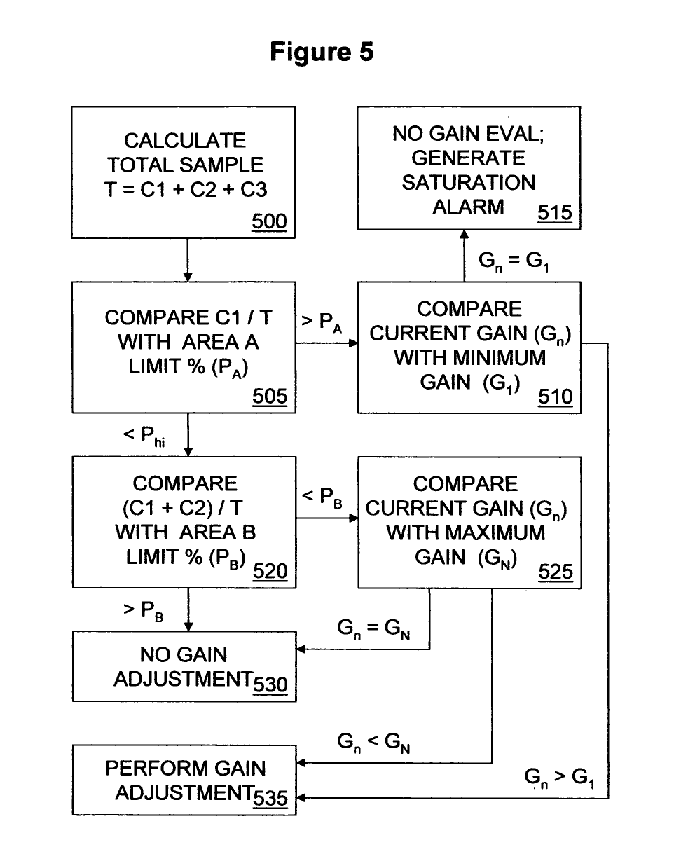

FIG. 5 shows a signal range check performed by acquisition system 110 under processor control attendant to gain reevaluation to determine whether gain adjustment is warranted, in some embodiments of the invention. Initially, acquisition system 110 calculates the total number of samples T taken in the sampling period as the sum of the three counts C1, C2 and C3 (500). A comparison is then made between the ratio CUT, which is the share of samples having amplitudes within domain A, and a limit share P_{A}, which is a maximum share of samples having amplitudes within domain A that will be tolerated without attempting gain adjustment (505).

If the ratio C1/T exceeds the limit share P_{A}, gain adjustment is indicated. Thus, a further comparison is made between the current gain Gₙ and a minimum gain G₁ to determine if it is possible to reduce gain (510). If the current gain Gₙ is higher than the minimum gain G₁, gain reduction is possible and gain adjustment is triggered (535). If the current gain G₁ is already set to the minimum gain G₁, gain reduction is not possible and gain adjustment is not triggered. Instead, a signal saturation alarm may be generated and sent to data output interface 115 (515).

If the ratio C1/T is below the limit share P_{A}, a further comparison is made between the ratio (C1+C2)/T, which is the share of samples having amplitudes within domain B, and a limit share P_{B}, which is a minimum share of samples having amplitudes within domain B that will be tolerated without attempting gain adjustment (520).

If the ratio (C1+C2)/T exceeds the limit share P_{B}, a gain increase is indicated. Thus, a further comparison is made between the current gain Gₙ and a maximum gain G_{N} to determine if it is possible to increase gain (525). If the current gain Gₙ is lower than the maximum gain G_{N}, a gain increase is possible and gain adjustment is triggered (535). If the current gain Gₙ is set to the maximum gain G_{N}, a gain increase is not possible and gain adjustment is not triggered (530).

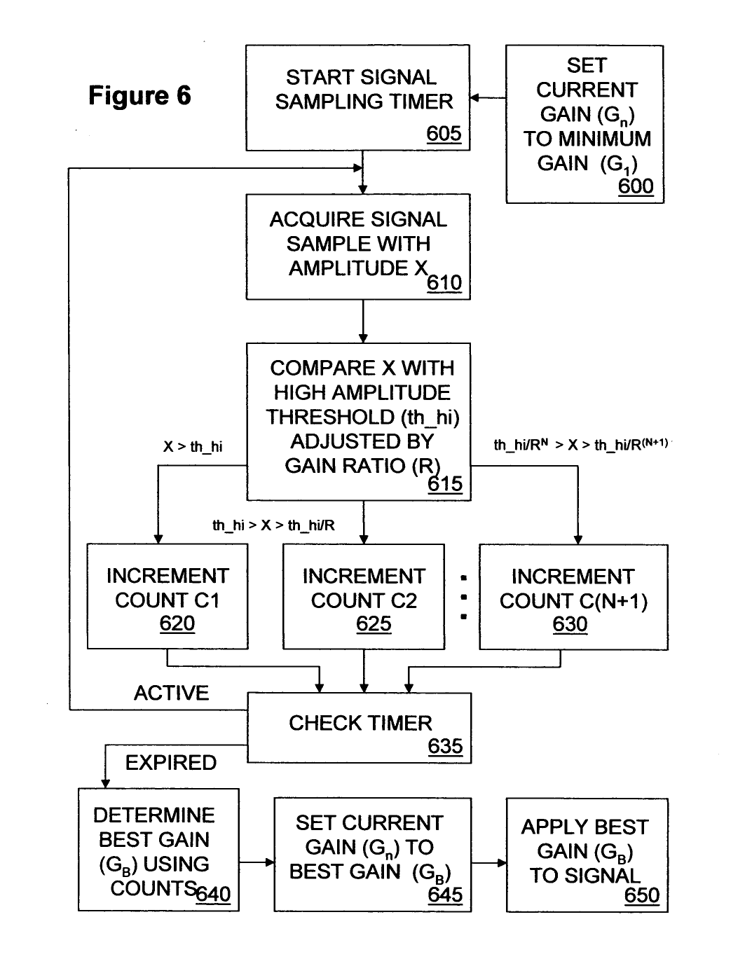

FIG. 6 shows gain adjustment performed by acquisition system 110 under processor control in some embodiments of the invention. Gain adjustment aims to select and apply a gain to the physiological signal that will keep the signal within a target amplitude range between the high amplitude threshold th_hi and low amplitude threshold th_lo in the current operating environment. Where monitoring system 100 supports N discrete gains, the gain is selected from a group of discrete gains G₁ to G_{N} where the gain ratio R=Gₙ/Gₙ₋₁ between consecutive gains is constant. Gain adjustment sampling is performed for a sampling period, after which the samples are analyzed and the best gain is determined. Initially, the current gain Gₙ is set to a minimum gain G₁ (600). At the beginning of the sampling period, acquisition system 110 starts a signal sampling timer (605) and acquires a first sample of the physiological signal, which sample has an amplitude X (610). The sample amplitude X is compared with a series of gain-specific amplitude ranges bounded by the high amplitude threshold th_hi as adjusted by exponentials of the gain ratio R (615) and an appropriate count between C1 and C(N+1) is incremented. If the sample amplitude X is above th_hi, a first count C1 is incremented (620). If the sample amplitude X is between th_hi and th_hi/R, a second count C2 is incremented (625). If the sample amplitude X is between th_hi/R^{N} and th_hi/R^{(N+1)}, a final count C(N+1) is incremented (625). More generally, if the sample amplitude X is between th_hi/Rⁿ and th_hi/R^{(N+1)}, a count C(n+1) is incremented. In any case, after incrementing an appropriate count, the timer is checked (635) and if the sampling period is still active, the flow returns to Step 605 and processes another sample. If the sampling period has expired, a best gain is determined (640) using the counts obtained in the sampling period, the current gain Gₙ is set to the best gain G_{B} (645) and the best gain is applied to the physiological signal (650).

The best gain is determined by acquisition system 110 under processor control as follows, in some embodiments of the invention. Initially, acquisition system 110 calculates the total number of samples T taken in the sampling period as the sum of all counts C1, . . . C(N+1). A comparison is then made between the ratio (C1+C2)/T, which is the share of samples having amplitudes within a gain-specific domain associated with a minimum gain G₁, and a limit share P_{L}, which is a threshold share of samples that will trigger selection of the gain associated with the current gain-specific domain as the best gain. If the ratio (C1+C2)/T exceeds the limit share P_{L}, gain G₁ is the selected as the best gain. If, on the other hand, the ratio (C1+C2)/T is below the limit share P_{L}, a comparison is made between the ratio (C1+C2+C3)/T, which is the share of samples having amplitudes within a gain-specific domain associated with the next lowest gain G₂, and the limit share P_{L}. If the ratio (C1+C2+C3)/T exceeds the limit share P_{L}, gain G₂ is the selected as the best gain. If the ratio (C1+C2+C3)/T is below the limit share P_{L}, additional comparisons are made between ratios having incrementally more counts and the limit share P_{L} until such a ratio is found to exceed P_{L}, at which point the gain associated with the ratio that exceeds P_{L} is selected as the best gain. More generally, comparisons continue until a ratio [C1+ . . . C(n+1)]/T is found to exceed P_{L}, at which point the gain Gₙ is selected as the best gain.

Acquisition system 110 makes a record of the gain has been applied to the physiological signal so that the original physiological signal can be later recovered. In some embodiments, gain records are transmitted by acquisition system to processing system 115 and the original physiological signal is recovered by processing system 115 before time domain signal processing is performed on the physiological signal.

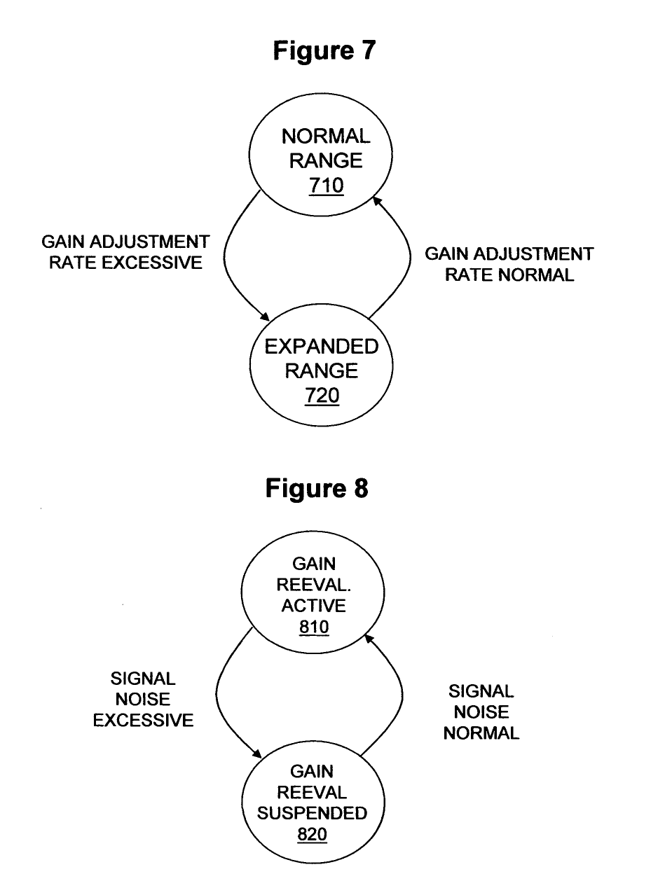

FIG. 7 shows dynamic range control performed by acquisition system 110 under processor control in some embodiments of the invention. Initially, the target amplitude range is a normal range having an upper bound at th_hi and a lower bound at th_lo (710). While in this normal state, the gain adjustment rate, i.e. the number of gain adjustments made in a given time period, is continually monitored and compared to a range expansion threshold. If the gain adjustment rate is detected above the range expansion threshold, the target amplitude range is expanded to an expanded range (720). For example, th_lo may be temporarily reduced to a smaller number, such as half of its normal value. While in this expanded state, the gain adjustment rate is continually monitored and compared to a range contraction threshold. If the gain adjustment rate is detected below the range contraction threshold, the amplitude range returns to the normal range (710). For example, th_lo may be returned to its original value.

FIG. 8 shows temporary gain reevaluation suspension performed by acquisition system 110 under processor control in some embodiments of the invention. Initially, gain reevaluation is active (810). While in this active state, the physiological signal is continually monitored for noise. If signal-to-noise ratio is detected below a threshold, the current gain is returned to its level when the signal was last detected to have a signal-to-noise ratio above the threshold and gain reevaluation is temporarily suspended (820). While in this suspended state, the physiological signal is continually monitored for noise. If signal-to-noise ratio is detected above a threshold, the temporary suspension is lifted and gain reevaluation is reactivated (810).

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. An automatic gain control (AGC) method for an ambulatory monitoring system, comprising the steps of:
continually monitoring, by the system, for gain reevaluation events;
reevaluating, by the system, a gain of a physiological signal in response to a gain reevaluation event, including sampling the physiological signal to obtain a first plurality of samples, determining a share of the first plurality of samples that is outside a target amplitude range and comparing the share with a first limit share; and
adjusting, by the system, the gain based at least in part on a determination that the share exceeds a first limit share.

2. The method of claim 1, wherein the gain adjusting step includes setting the gain to a minimum, sampling the physiological signal to obtain a second plurality of samples, determining shares of the second plurality of samples that are within respective gain-specific amplitude domains and adjusting the gain to a level associated with a gain-specific amplitude domain for which a share of the second plurality of samples exceeds a second limit share.

3. The method of claim 1, wherein the gain reevaluation events include function change events.

4. The method of claim 1, wherein the gain reevaluation events include motion sensor events.

5. The method of claim 1, wherein the gain reevaluation events include periodic signal check events.

6. The method of claim 1, further comprising the step of adjusting, by the system, the target amplitude range in response to a determination that a gain adjustment rate exceeds a limit rate.

7. The method of claim 1, further comprising the step of temporarily suspending, by the system, the gain reevaluating and gain adjusting steps in response to a determination that a noise level of the physiological signal exceeds a limit noise level.

8. An AGC method for an ambulatory monitoring system, comprising the steps of:
reevaluating, by the system, a gain of a physiological signal including comparing the physiological signal with a target amplitude range;
adjusting, by the system, the gain in response to the comparison; and
adjusting, by the system, the target amplitude range in response to a determination that a gain adjustment rate exceeds a limit rate.

9. The AGC method of claim 8, further comprising the step of temporarily suspending, by the system, the gain reevaluating and gain adjusting steps in response to a determination that a noise level of the physiological signal exceeds a limit noise level.

10. The AGC method of claim 8, further comprising the step of continually monitoring, by the system, for gain reevaluation events.

11. The AGC method of claim 10, wherein the gain reevaluation events include function change events.

12. The AGC method of claim 10, wherein the gain reevaluation events include motion sensor events.

13. The AGC method of claim 10, wherein the gain reevaluation events include periodic signal check events.

14. the AGC method of claim 8, wherein the reevaluating step includes sampling the physiological signal to obtain a plurality of samples, determining a share of the plurality of samples that is outside a target amplitude range and adjusting the gain based at least in part on a determination that the share exceeds a limit share.

15. The AGC method of claim 8, wherein the gain adjusting step includes setting the gain of the physiological signal to a minimum, sampling the physiological signal to obtain a plurality of samples, determining shares of the plurality of samples that are within respective gain-specific amplitude domains and adjusting the gain to a level associated with a gain-specific amplitude domain for which a share of the plurality of samples exceeds a limit share.

16. An AGC method for an ambulatory monitoring system, comprising the steps of:
reevaluating, by the system, a gain of a physiological signal including comparing the physiological signal with a target amplitude range;
adjusting, by the system, the gain in response to the comparison; and
temporarily suspending, by the system, the gain reevaluating and gain adjusting steps in response to a determination that a noise level of the physiological signal exceeds a limit noise level.

17. The AGC method of claim 16, further comprising the step of adjusting, by the system, the target amplitude range in response to a determination that a gain adjustment rate exceeds a limit rate.

18. The AGC method of claim 16, further comprising the step of continually monitoring for function change events, motion sensor events and periodic signal check events.

19. the AGC method of claim 16, wherein the reevaluating step includes sampling the physiological signal to obtain a plurality of samples, determining a share of the plurality of samples that is outside a target amplitude range and adjusting the gain based at least in part on a determination that the share exceeds a limit share.

20. The AGC method of claim 16, wherein the gain adjusting step includes setting the gain of the physiological signal to a minimum, sampling the physiological signal to obtain a plurality of samples, determining shares of the plurality of samples that are within respective gain-specific amplitude domains and adjusting the gain to a level associated with a gain-specific amplitude domain for which a share of the plurality of samples exceeds a limit share.
